(19) United States
(12) Patent Application Publication
(10) Pub. No.: US 2007/0238146 A1
(43) Pub. Date: Oct. 11, 2007

(54) Kinetic metabolic assay for antifungal susceptibility testing

(76) Inventors: Bonnie J. Tyler, Salt Lake City, UT (US); Prasanna Khot, Mountlake Terrace, WA (US)

Correspondence Address: TRASK BRITT, P.O. BOX 2550, SALT LAKE CITY, UT, 84110, US

(21) Appl. No.: 11/706,632

(22) Filed: Feb. 14, 2007

Related U.S. Application Data
(60) Provisional application No. 60/773,663, filed on Feb. 16, 2006.

(51) Int. Cl. C12Q 1/04

(52) U.S. Cl. 435/34

(57) ABSTRACT

Methods for determining viable cell numbers and percentages are provided. The kinetic profile of a metabolic indicator associated with the cells may be analyzed to determine the number of viable cells in the starting population. Cells may be cultured with a molecule of interest and the effect of that molecule on the kinetic profile can be related to the susceptibility of the cells to the molecule of interest.

### CROSS-REFERENCE TO RELATED APPLICATION

Claim of Priority: Pursuant to the provisions of 35 U.S.C. § 119(e), this application claims the benefit of the filing date of Provisional Patent Application Ser. No. 60/773,663, filed Feb. 14, 2006, for “KINETIC METABOLIC ASSAY FOR ANITMICROBIAL SUSCEPTIBLITY TESTING”, the contents of the entirety of which are incorporated herein by this reference.

### GOVERNMENT RIGHTS

This invention was made with government support under DE13231-02 awarded by the NIH. The Government has certain rights to this invention.

### FIELD OF THE INVENTION

Field of the Invention: The present invention relates generally to metabolic assays. More particularly, the present invention relates to highly quantitative metabolic assays for studying metabolic rate, proliferation rate, and drug susceptibility.

### BACKGROUND

In recent years, a wide spectrum of medical conditions and treatments cause patients to become immunocompromised. This has led to a dramatic increase in the occurrence of invasive fungal infections (13, 34). Both intrinsic and acquired resistance during antifungal therapy is now recognized as a major clinical problem (34). Several drugs with varying mechanisms of action have been introduced for antifungal therapy. As a consequence, antifungal susceptibility testing (AST) has gained prominence due to its role in guiding therapy and aiding the drug development process (13, 33, 36). Predicting clinical outcome from susceptibility testing remains a challenge. This problem persists because current in vitro susceptibility testing techniques fail to capture the complexities of the host and the pathogenic state of infection (19, 36, 42).

The biofilm paradigm has emerged as a pertinent model for many fungal and bacterial infections. It has been estimated that biofilms cause over 2 million infections annually in the United States resulting in an estimated $ 11 billion additional patient health care cost (31). From a rigorous clinical perspective, in vitro biofilm models are most appropriate for testing hypotheses related to biomaterial-centered infections. However, from a broader, fundamental perspective, their utility for advancing understanding of infective fungal communities extends further. In general, microbial biofilms exhibit an exceptional ability to survive doses of antimicrobial agents that are many times greater than the dose which is lethal for their planktonic counterparts (6, 8, 24, 44). Fungal biofilms like those of Candida albicans have been shown to be resistant to a variety of azoles, including fluconazole (15), voriconazole (25), miconazole (22), itraconazole (15), ketoconazole (15), the antiseptic chlorhexidine (4, 5), flucytosine (15), and the polyenes nystatin (4, 5), and amphotericin B (AmB) (4, 5, 15, 39, 40).

Metabolic assays based on reduction of XTT [2,3-bis(2-methoxy-4-nitro-5-sulfor-dulfophenyl)-5-[(phenylamino)carbonyl]-2H-tetrazolium hydroxide] or alamarBlue are emerging as a promising choice of AST (10, 16, 28, 38, 45, 48). These assays can be adapted for in situ testing which captures some of the complexities of the pathogenic state of the organism (39). In addition, these assays are increasingly preferred for systems where methods based on turbidity or plate counts provide inconclusive results (2, 7, 39, 46). This is especially the case for susceptibility testing of biofilms and mycelium forming multicellular fungi (23, 26, 39). Moreover, metabolic assays can be readily adapted for rapid and high throughput AST giving them a notable edge over conventional methods (1, 35).

Development of metabolic assays aimed at efficiently replacing conventional minimum inhibitory concentration (MIC) tests have naturally adopted a model whereby a threshold concentration of antifungal agent that results in no viable cells is determined (10, 32, 37, 48). This model has also been adapted in cases in which the metabolic assay is used for in situ analysis of biofilms (5, 35, 39). However, in applying metabolic assays for obtaining data that may elucidate the reasons for biofilm resistance it is useful to know, not only the threshold concentration of antifungal agent that inactivates the cells, but also the proportion of the cell population that remains viable after exposure to an antifungal agent. It is this subpopulation that is most likely responsible for the observed transient resistance of biofilms (3, 18, 35, 39), and its size can be a critical component of understanding the mechanism of this transient resistance phenomenon (21). Thus it would be an advancement in the art to develop metabolic assays which not only determine a minimum effective concentration for a molecule of interest, but also the proportion of viable cells present after treatment with such molecule.

### BRIEF SUMMARY OF THE INVENTION

Methods of determining susceptibility of a biofilm to a molecule of interest are provided. One embodiment of a method comprises culturing the biofilm with the molecule of interest, determining the kinetic profile of a metabolic indicator associated with the biofilm, determining the number of viable cells in the biofilm, and determining the percentage of surviving cells in the biofilm. Determining the number of viable cells in the biofilm may comprise analyzing an exponential phase of the kinetic profile of the metabolic indicator.

Methods of determining the number of viable cells in a culture are also provided. One embodiment of a method comprises determining the kinetic profile of a metabolic indicator associated with the culture and determining the number of viable cells in the culture. Determining the number of viable cells in the culture may comprise analyzing the kinetic profile the metabolic indicator.

### BRIEF DESCRIPTION OF THE DRAWINGS

In order to illustrate the manner in which the above recited and other advantages of the invention are obtained, a more particular description of the invention briefly described above will be rendered by reference to specific embodiments thereof which are illustrated in the appended drawings. As these drawings only depict embodiments of the invention, and are not limiting of its scope, the invention will be described and explained with additional specificity and detail through the use of the accompanying drawings in which.

FIG. 1 is graphical representation of percent reduction of alamarBlue over time by C. albicans in exponential phase. Different initial concentrations of viable cells are (∇) 8×10⁶; (*) 2.2×10⁶; (⋄) 5.7×10⁵; (□) .1.6×10⁵; ( [symbol] ) 4.0×10⁴; ( [symbol] ) 1.7×10³; (0) 7.8×10²; ( [symbol] ) 1.5×10²; (-·-) 0.0×10¹; and (−) no cells.

FIGS. 2(a) and 2(b). FIGS. 2(a) and 2(b) are graphical representations of linear calibrations for a kinetic assay using alamarBlue as an indicator the calibrations apply over a dynamic range of approximately six orders of magnitude (10¹ to 10⁷ CFU). FIG. 2(a) is a calibration using the curve fitting technique (R²=0.983); FIG. 2 (b) is a calibration using the curve fitting technique (R²=0.983); FIG. 2(b) is a calibration using the curve (R²=0.991). Similar calibrations were obtained for all combinations of variables shown in Table 1.

FIG. 3 is a graphical representation of the linear dynamic range when metabolic assays are run as endpoint assays. The linear dynamic range is approximately two orders of magnitude. Percent reduced alamarBlue is shown for a range of initial viable cells plotted at endpoints of (●) 300 min.; (▪) 600 min.; (▴) 900 min.; and (X) 24 hours. Similar profiles were obtained using XTT and turbidity as metabolic indicators.

FIGS. 4(a)-4(d) are graphical representations of comparisons of Kinetic Metabolic Assays (KMAs) using XTT or alamarBlue with direct CFU measurements. (⋄) is alamarBlue via curve fitting; (−) is alamarBlue via t-threshold; (▴) is XTT via curve fitting; (−) is XTT via t-threshold; and (−) is CFU. FIG. 4 (a) is cells in exponential phase and modified medium; FIG. 4(b) is cells in stationary phase and modified medium; FIG. 4 (c) is cells in exponential phase and unmodified medium; and FIG. 4 (d) is cells in stationary phase and unmodified medium.

FIG. 5 is a graphical representation of the effect of modified growth medium on correlations between CFU and kinetic assays. Presented are AmB dose response curves for the combination of t-threshold technique, exponential-phase cells, and alamarBlue as the metabolic indicator. Data from three independent experiments is shown with (±) standard errors. (▴) is alamarBlue with unmodified medium; (▪) is alamarBlue with modified medium; and (●) is CFU.

FIG. 6 is a graphical representation of differences in AmB susceptibility between expontial phase and stationary phase C. albicans. AmB dose response curves for the t-threshold technique using alamarBlue as the metabolic indicator are presented. Data from three independent experiments is shown with (±) standard errors. (▴) is cells in stationary phase; and (▪) is cells in exponential phase.

FIG. 7 is a graphical representation of the susceptibility of C. albicans biofilm treated with AmB with percent viability estimated using an alamarBlue based KMA. The susceptibility of the of the basal blastospores subpopulations was tested in situ. The percent viability ± propagated standard deviation was generated from three separate experiments. (●) is basal blastospores; (▴) is shear removed biofilm; (□) is stationary phase planktonic cells; and (*) is exponential phase planktonic cells.

FIG. 8 is a graphical representation of kinetic curves of the reduction of alamarBlue for samples untreated with AmB, i.e. the inoculum (or positive control) of the samples in FIG. 7. (●) is basal blastospores; (▴) is shear removed biofilm; (□) is stationary phase planktonic cells; (*) is exponential phase planktonic cells; and ( ) is blank.

### DETAILED DESCRIPTION

One embodiment of the present invention describes a method of using a kinetic metabolic assay (KMA) for quantifying viable cells in a sample based on analysis of the kinetics of change in or state of (the kinetic profile) a metabolic indicator. In embodiments of the invention, a KMA may be used to quantify a viable cell population over a range of 10¹ to 10⁷ cells.

As will be apparent to one of ordinary skill in the art any metabolic indicator that may be associated with cells may be used, such as but not limited to, turbidity, fluorescent dyes, and redox indicators such as, but not limited to, alamarBlue and XTT. Metabolic indicators may be components inherent to the cells or components added to the environment of the cells. In embodiments of the present invention, changes in or the state of the metabolic indicator may result in alteration of ability of the media containing the sample to absorb or reflect particular wavelengths of radiation. In further examples, the absorbance may be read at wavelengths of, for example, but not limited to, 492 nm, 570 nm, 600 nm, and combinations thereof.

In embodiments of the present invention, a kinetic profile of the metabolic indicator may be determined. Determining a kinetic profile may comprise recording the state of or change in the metabolic indicator over time. A series of plots of the state of or change in the kinetic indicator may comprise the kinetic profile. Portions of the kinetic profile may comprise kinetics that may be described as, but are not limited to, exponential; saturation kinetics like Monod kinetics and its variant forms; linear; polynomial (for example, but not limited to, zero, first, or second order). As will be apparent to one of ordinary skill in the art, the exact kinetics of the kinetic profile are not important that that viable cell numbers or a percentage of viable cells can be determined as long as the kinetic profile is predictable and/or reproducible for a particular set of parameters.

In embodiments of the present invention, quantifying viable cells may be based on analysis of an exponential portion of the kinetic profile of the metabolic. In embodiments of the invention, change in or state of the metabolic indicator may be used to generate a kinetic profile, such as, but not limited to, a curve of data points representing change in or state of the metabolic indicator over time. In additional embodiments, data points to generate a kinetic profile may be acquired from about every 1 minute to about every 30 minutes; from about every 5 minutes to about every 15 minutes, or about every 10 minutes. In further embodiments, analysis of the kinetic profile determine percent viable cells may be performed by, for example, but not limited to, fitting the profile to an exponential model (curve fitting) or by using the time to reach a fixed threshold (t-threshold) of change in or state of the metabolic indicator. In further embodiments, acquisition and/or analysis of the kinetic profile may be facilitated by computational means, such as, but not limited to, Matlab® and/or KC4™ software.

In embodiments of the invention, acquisition and/or analysis of the kinetic profile of the metabolic indicator may be automated. Examples of devices which may be used to automatically acquire and/or analyze the kinetic profile of the metabolic indicator include, but are not limited to, plate readers such as a heated plate readers, a shaking plate readers, and/or combinations there of such as a Biotek Synergy-HT™ plate reader.

In embodiments of the invention, the cells subjected to a KMA may be any kind of cell, such as, but not limited to, prokaryotic, eukaryotic, bacterial, fungal, plant, animal, mammalian, gram-negative, gram-positive, human, C. albicans, cells and mixtures thereof. In further embodiments of the present invention, the cells subjected to a KMA may be in any state relative to growth environment, such as, but not limited to, floating, planktonic, adherent, part of or associated with a biofilm or other cell clusters or masses, which may or may not be adhered to or otherwise associated with a solid surface, and which may or may not contain hyphae, and mixtures thereof. In additional embodiments, a KMA analysis of a biofilm may be performed in situ, without disrupting the film or removing the film from a backing or solid support to which it may have adhered. In further embodiments of the present invention, cells subjected to a KMA may be for example, but not limited to, stationary, logarithmic, exponential, senescent, proliferative, transformed, and/or immortal, and mixtures thereof

In embodiments of the present invention, a KMA may be calibrated by comparison of the percent viable cells as determined by KMA with percent viable cells determined by a second method, such as, but not limited to, turbidity, total protein levels, specific protein levels, DNA assays, cell counts such as those using a hemocytometer or coulter counter and/or a determination of Colony Forming Units (CFU). The output of the KMA assay may then be subjected to an influence, such as, but not limited to, a correction factor, so as to more accurately represent a percentage of viable cells.

Further embodiments of the invention provide include methods for susceptibility testing. In embodiments of the invention, a sample of cells may be treated with a molecule of interest for various periods and at various concentrations. The sample of cells may then be subjected to a KMA to determine if there has been a change in the percentage of viable cells in the sample.

In embodiments of the invention, the molecule of interest may be beneficial or detrimental to the cells in a sample. Examples of molecules of interest that may be beneficial to cells in a sample, include, but are not limited to, nutrients, growth factors, vitamins, minerals, hormones, cytotoxic compounds, antibiotics, and combinations thereof Examples of cytotoxic compounds and antibiotics include, but are not limited to Penicillin G; D-Cyloserine; Vancomycin; Bacitracin; Cephalosporin C; Tetracycline; Erythromycin; Chloramphenicol; Streptomycin; Nalidixic Acid; Rifampicin; Triethoprim; EDTA; and Lysozyme. Examples of antifungals include, but are not limited to, Amphotericin B; Nystatin; Caspofungin; Mycafungin; Anidulafungin; Fluconazole; Intraconazole; Voriconazole; Posaconazole; Chlorhexidine; Terbinafine; Flucytosine; and Gaiseofulvin.

In additional embodiments of the present invention, the molecule of interest may be present during a KMA or may be removed from the sample before a KMA. In further embodiments, molecules or compounds known to inhibit or affect the activity of the molecule of interest may be added to the sample before or during a KMA. Examples of compounds known to affect the activity of AmB include for example, but not limited to, ergosterol, KCl and MgCl₂, (11, 12, 17). In embodiments, the sample may be placed in fresh medium or other solution before subjecting the sample to a KMA. In other embodiments, the reagents for KMA may be added directly to the sample without first washing the sample.

In further embodiments of the invention, the cell sample may be a fungal biofilm, such as but not limited to, C. albicans, and the molecule of interest may be an antifungal, such as, but not limited to, polyenes such as amphotericin B (AmB). C. albicans is the most prominent opportunistic fungal pathogen in humans and AmB a common antifungal agent of the polyene class (9, 43). The calibration of a KMA in predicting viability of AmB treated cells may be performed by correlating the KMA results with a kinetic method that relies on turbidity and/or with direct CFU measurements.

Embodiments of the present invention provide a method for antimicrobial susceptibility testing of planktonic (free floating) and biofilms (adhered) of fungi or bacteria when exposed to antimicrobial agents.

The ability of the KMA to quantitatively detect small subpopulations of surviving cells is particulary relevant for susceptibility of biofilms. In studies elucidating reasons for biolfilm resistance, it is useful to know, not only the threshold concentration of the antifungal that inactivates the cells, but also the proportion of the cell population that survives, since it is this small subpopulation that is most likely responsible for resistance. Moreover, in biofilm susceptibility testing, there is lack of quality control of the ratio of the active drug to cell mass (or numbers), which is known to affect drug susceptibility results (12). The exceptionally high minimum inhibitory concentration (MIC) values for biofilms often reported may simply be an effect of high cell mass rather than its true level of resistance (4, 15, 35, 39). A KMA controls for inoculum size by enabling the precise quantification of the biofilm inoculum in silu. In addition, the KMA inherently normalizes the heterogeneous metabolic states of a biofilm because of its dependence on post drug treatment cell proliferation to reduce the metabolic indicator. This is achieved by forcing the surviving cells to proliferate at the same growth rate independent of their initial metabolic state.

The in situ susceptibility testing for biofilms is a promising approach to minimize the discrepancies between in vitro antifungal susceptibility data and in vivo clinical outcomes of biofilm infection (41, 43). Since the resistance of biofilms at least partially originates from being in a biolfilm environment; it is vital to use an in situ technique like the KMA which is non-destructive instead of solid agar based methods. In addition, the KMA can be used on systems containing large cell clusters, biofilms and/or hyphae which invalidate plate counts or assays based on trubidity.

In describing and claiming the present invention, the singular forms “a,”“an,” and “the ” include plural referents unless the context clearly dictates otherwise.

The present invention is further described in the following examples, which are offered by way of illustration and are not intended to limit the invention in any manner.

### EXAMPLE

### Example 1

### Materials and Methods

C. albicans strain and medium. C. albicans CA-1 isolate obtained from the culture collection of Diane Brawner (Microbiology Department, Montana State University)(14). The strain was stored at −80° C. Planktonic cells were cultured in 2% YEPD medium (2% glucose, 1% bacto yeast extract, and 2% bacto peptone). The solid agar medium for the CFU assay was was 1% glucose, 0.5% bacto yeast extract, 2% bacto agar, 0.1% ammonium sulfate dissolved in 20% tap water and 80% D.I. water.

Modified growth medium. In addition to the 2% YEPD medium, the modified growth medium contained optimal concentrations of ergosterol (Alfa Aesar; catalog no. 57-87-4), MgCl₂(Sigma; catalog no. M4880), and KCl (Sigma; catalog no. P5405). The final concentrations of these reagents in the kinetic assays were 40 μM ergosterol, 88 μM MgCl₂, and 42.5 μM KCl.

CFU assay. CFU were estimated for both AmB treated and untreated planktonic sample. 100 μl planktonic C. albicans cells in PBS were serially diluted in 2 ml cuvettes (catalog no. BTCUV, Biotrace Inc.). The serial dilution was 10-fold across each cuvette. The required number of serial dilutions per sample were judged based on trial and error. A volume of 100 μl from each serially diluted cuvette was spread as a separate lane on an agar plate. Each agar plate lanes of serial had a maximun of 4 lanes. Plates were incubated at 37° C. for 24 hours. CFU were estimated from dilutions whose numbers fell in the range of 10 to 100 colonies per lane. The appropriate dilution factor was multiplied to estimate the final viable cell concentration for every sample.

Planktonic cultures. Cultures were grown aerobically in 250-ml Erlenmeyer flasks containing 100 ml growth medium. The flasks were placed in a shaker incubator at 37° C. and 160 rpm for the desired period of growth. C. albicans grew as budding yeast under these conditions. Exponential phase cells were harvested after a 5-h growth period and stationary-phase cells at 96 h.

Biofilm cultures. Biofilms were grown in a tubular flow cell (TF). Silicone tubing (Cole-Panmer; catalog no. EW-95802-08) with an inner diameter of 4.78 mm, an outer diameter of 6.35 mm, a wall of 0.79 mm, and a length of 60 cm constituted the biofilm-growing region of the TF. The source of growth medium for the TF was a 2-liter Erlenineyer flask. A bubble trap was placed between the Erlenmeyer flask and the TF to prevent passage of air bubbles during biofilm growth. Flow rates of 1.17 ml/min (shear rate, 109.5 s⁻¹) were maintained by a peristaltic pump coupled at the effluent end of the TF. The residence time for the volume of liquid contained in the tubular reactor portion of the flow system (20-cm length of tubing) was 3 min. This condition ensured that the contribution to the cell population in the TF from cells in the planktonic mode of growth (doubling time, approximately 80 min) was negligible. After sterilization by autoclaving, the entire setup was placed horizontally on a grilled shelf in an incubator at 37° C. The TF was filled with growth medium before being inoculated with cells. The inoculum was prepared from a 24-h planktonic culture at a concentration of 10⁸ cells/ml in 0.1 M phosphate-buffered saline (PBS, pH 7) buffer. It was fed into the TF from the effluent end by reversing the direction of flow. Flow was then discontinued for 1 h. After the 1 -h inoculation period, flow was resumed for 36 h. At the end of this culture period, the section of tubing in which the biofilm grew was clamped at both ends and removed by cutting the tubing with a sterile blade. The liquid column was drained into a petri dish by moving the tubing to a vertical position and releasing the clamps. The tubing was then rinsed with PBS buffer equivalent to one tube volume. The fraction of biofilm collected in the petri dish by this procedure is referred to as the shear-removed biofilm. The cells that remained adhering to the walls of the tube are referred to as the basal blastospores or the basal blastospore subpopulation.

AmB treatment. AmB was from Biosource International Inc. (Fungizone with 0.00205% sodium deoxycholate solubilizing agent). A standard broth dilution method was used to assess the AmB MlC of CA-1, with ATCC 24433 used as a reference strain (32). AmB treatment of planktonic cells and shear-removed biofilm was preformed in 1.5-ml centrifuge tubes. The tubes contained a total working volume of 450 μl. AmB dissolved in 200 μl of 0.1 M PBS (pH 7.0), and cells resuspended in 250 μl of PBS adjusted to an optical density of 0.05 (A₆₆₀) were added to the 1.5-ml tube. After more than 10 preliminary experiments, the time between recovery of the shear-removed biofilm in the petri dish and exposure to AmB was reduced to approximately 5 min. The positive controls had no AmB, and the negative controls had no cells. For AmB treatment of the basal blastospore population, each sample consisted of a 0.5-cm long section of tubing cut from the TF. Each tubing sample was positioned in the 96-well plate (Corning Inc.; Costar 3370) such that its outer wall snugly fit along the wall of the well in the 96-well plate. This enabled recording of absorbance data for the metabolic indicator in real time without perturbing the well contents. The 0.5-cm tubing samples were randomly placed in the 96-well plate in order to randomize the distribution between treated samples and untreated controls. Solution (225 μl) containing AmB in 0.1 M PBS was added to wells to completely submerge the 0.5-cm tubing during the AmB treatment phase. Untreated samples were the positive controls. Three independent TF experiments were run, generating 24 samples per AmB concentration and 61 untreated controls for the basal blastospores. Sterile tubing was used as a negative control. In every experiment, a minimum of four negative controls were used. The AmB treatment period was 1 h across all cell populations. During AmB exposure, the 1.5-m1 tubes (containing either the planktonic or shear-removed populations) and 96 -well plates (containing the basal blastospores) were placed in a shaker incubator at 37° C. and 150 rpm. After AmB treatment, cells in 1.5-ml tubes were centrifuged for 5 min at 4,000 Xg, the supernatant was decanted, and the pellet was resuspended in the same volume of fresh PBS buffer. Cells were then transferred into wells of the 96 -well plate system for the metabolic assay. For the 96-well plate containing sections of tubing, AmB was carefully aspirated out of the wells and immediately replaced with reagents of the metabolic assay.

Kinetic curves. Kinetic data of the reduction of metabolic indicators and turbidimetric changes were recorded for both AmB treated and untreated samples. The components of the kinetic metabolic assay were at the same concentrations for all populations tested. Kinetic data was generated for XTT (Sigma; catalog no. X4626), alamarBlue (BioSource International; catalog no. DAL1100) and turbidity (optical density of biomass). Final concentration of menadione (catalog no. M5625) at 1 μM was used as an electron coupling agent with 0.05 mg/ml XTT. Each well had a total working volume of 230 μl. It contained 100 μl of cells in PBS from a sample, 25 μl metabolic dye, 75 μl of a PBS solution with AmB quenching reagents (modified medium) or without them (unmodified medium) and, 30 μl 2% YEPD growth medium. In addition, antibacterial reagent (penicillin-streptomycin; Invitrogen catalog no. 15140-122) at a final concentration of 1% by volume was used in each well. The kinetic assay in the 96 well format was run for 24 h at 37° C. with continuous shaking in a Synergy-HT plate reader (Biotek Inc.).

Absorbance data was recorded at 492 nm for the XTT, at 570 nm and 600 nm for the alamarBlue dye and 660 nm for turbidity. Data was collected every 10 min for 24 hours, generating 140 data points per sample. Each experiment resulted in an array of kinetic data corresponding to samples in the 96 well plate. Data was generated from three independent experiments. Kinetic data was exported from the KC4™ software (Biotek Inc.) into Microsoft Excel®. Matlab® codes specific to the calculation requirements of each metabolic indicator, or turbidity, were used to automate data analysis. The percent reduction of the alamarBlue indicator was calculated as per the manufacturer's formula (product literature; BioSource International Inc.; catalog no. DAL1100). For the XTT indicator, final reduction of the dye was estimated by subtracting the absorbance value of 492 nm with the absorbance value at 660 nm. The value at 660 nm served as a reference for the XTT reduction and, also a measure of the turbidity.

Data Analysis

(i) Calibration of the kinetic assays. For the most effect use of kinetic analysis, the exponential portion of the metabolic curve is analyzed. Equations (1) and (2) represent the exponential portion of a sigmoidal kinetic curve.
X_{F}=X₀exp(μt) (1)
ln(X₀)=ln(X_{F})−(μt) (2)
In equations (1) and (2), X_{F} and X₀ are cell or metabolic indicator concentrations at set threshold time (t) and at initial time (t=0) respectively. μ is the specific growth rate of cells. Eq. (2) is a linearized version of Eq. (1). To correlate a kinetic parameter with viability, two techniques of data analysis were explored. The first approach (called t-threshold) involved estimating the time required for a specific number of initial viable cells to reach a fixed threshold of reduction. The threshold value was chosen such that, irrespective of the number of initial viable cells; every sample would be in its exponential phase of metabolic reduction at the set threshold. The time taken to reach a set threshold of reduction correlated linearly with the natural logarithm of the initial cell number. This linear relationship is apparent in Eq. (2). The second approach (called curve fitting) involved fitting an exponential model (Eq. (1)) to the exponential part of the kinetic data (up to approximately 65% reduction or turbidity). The least squares algorithm was used for curve fitting. The curve fitting procedure yields two constants X₀ and μ. From Eq. (2) it is evident that the natural logarithm of X₀ correlates linearly with the natural logarithm of initial cell numbers.

Matlab® codes were written to estimate the kinetic parameters of each assay. For the t-threshold technique, linear interpolation was used to estimate the precise time taken by each sample to reach the fixed threshold. The threshold values for each kinetic assay are shown in Table 1. The curve fitting technique involved using kinetic data up to a set threshold. Matlab®'s curve fitting toolbox was used to fit the selected data to an exponential model (Eq. (1)). The value of the constant from the fit was accepted only if the R² value (the goodness of fit) exceeded 0.98.

TABLE 1
Threshold values for the kinetic data analysis techniques.

| Kinetic indicator | T-threshold technique | Curve fitting technique |
|---|---|---|
| alamarBlue | 50% reduced | 70% reduced |
| XTT | 0.35 A_{492-660 nm} | 0.45 A_{492-660 nm} |
| turbidity | 0.35 A_{660 nm} | 0.45 A_{660 nm} |

The kinetic parameters for each combination of data analysis technique and kinetic indicator were estimated for initial viable cells that ranged from approximately 10¹to 10⁷CFU. The resulting linear plot formed the basis of calibrating the kinetic assays in terms of CFU.

(ii) Calculation of percent viability. Viability in terms of CFU was calculated by using the kinetic parameters estimated for each sample and the corresponding calibrations. The viable fraction for every kinetic method or CFU assay was estimated as a percentage of its corresponding positive control (no AmB treatment). Dose response curves contained percent viability plotted for increasing AmB concentrations. All data was mean percent viability from three independent experiments ± standard error.

(iii) Correlation between assays. To estimate the correlation in predicting viability between two independent assays, a Pearson's correlation coefficient (two-tailed) was estimated using Microsoft Excel® (30, 35). Data between AmB concentration>0 (i.e. the 100% viability data points were excluded) until AmB concentration that first corresponded to 0% viability were used. These estimated the most conservative levels of correlations.

### Example 2

### Time Course of Metabolic Reduction

Metabolic assays are either performed during (10, 37, 48), or after treatment of the antifungal agent (1, 5, 45). In either case these assays measure the degree of metabolic reduction by endpoint analysis (5, 20, 39, 45). KMA presents an alternative approach in which, for example, the reduction of the metabolic indicator is followed in real-time. The KMA enhances the dynamic range up to six orders of magnitude by taking advantage of cell proliferation to amplify viable cells.

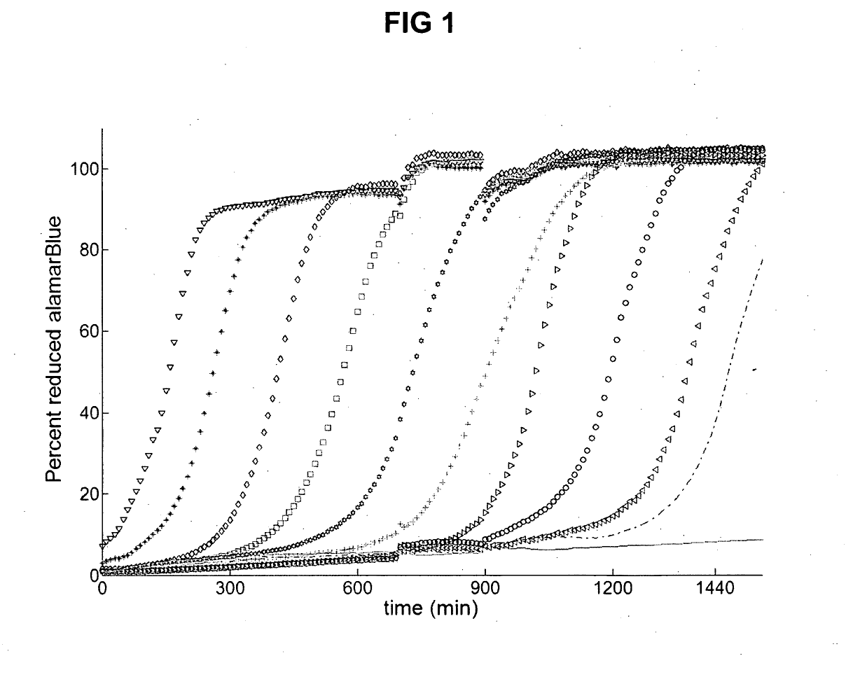

The kinetic curves of metabolic reduction of XTT and alamarBlue followed a sigmoidal shape (FIG. 1). The corresponding curves of turbidimetric changes had an overlapping profile implying that metabolic reduction was primarily occurring due to cell proliferation. This was expected because all cells were placed in a fresh growth medium after drug treatment by isolating them from the solution with drug. FIG. 1 shows the kinetic curves for the alamarBlue assay for a wide range of initial inoculum (10 to 10⁷ CFU). Similar curves were obtained with XTT and turbidity. The curves proportionally shift along the time axis as the size of the initial inoculum changes, thus forming the basis of a wide dynamic range. This effect could be simulated using Eq. (1) (data not shown). The exponential portions of the kinetic curves shown in FIG. 1 constitute the region from 0% to approximately 65% reduction of alamarBlue. The exponential portion of each kinetic curve fits Eq. (1) resulting in R² values that are>0.98. These fits applied to all kinetic indicators (alamarBlue, XTT and turbidity). This implies that the exponential equations (Eq. (1) and (2), were adequate representations of the chosen experimental system.

### Example 3

### Calibration and Dynamic Range of the KMA

The KMA based on XTT or alamarBlue were calibrated with CFU. An additional assay based on the same kinetic principle was developed using turbidimetric changes as an indicator. As a result, four assays were simultaneously applied to every planktonic sample. Three assays constituted kinetic analysis and the fourth was a CFU assay. In addition, two data techniques curve fitting and t-threshold) were used to analyze data from each kinetic assay. The kinetic assays were performed in the presence of an unmodified- and also a modified 2% YEPD medium designed to quench the action of residual AmB after treatment. C. albicans chosen for all the calibration experiments were from two planktonic growth phases, exponential- and stationary-phase cells.

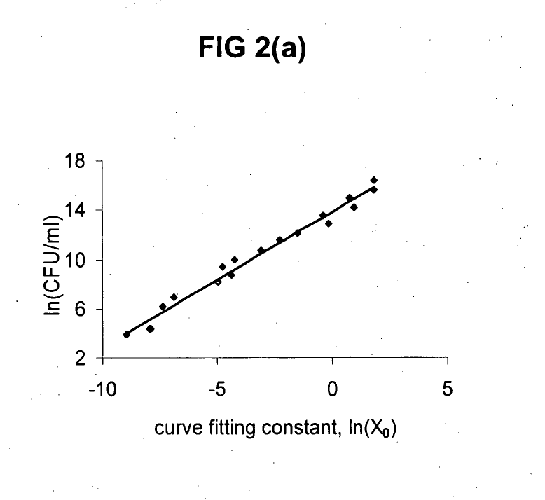

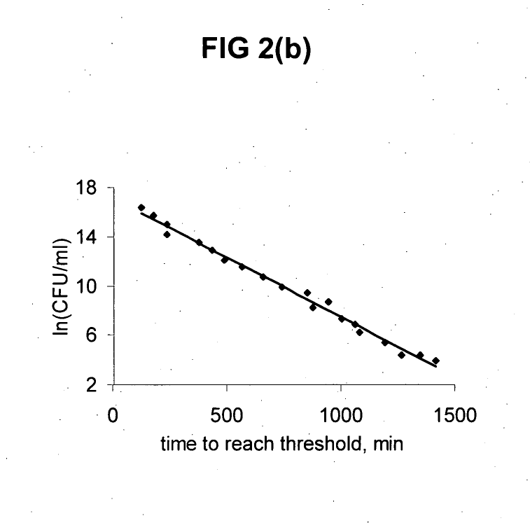

Calibrations were constructed for all combinations of variables (Table 2). FIG. 2a and 2b show an example of the calibrations constructed for the alamarBlue indicator using both techniques of data analysis i.e., curve fitting (FIG. 2a) and t-threshold (FIG. 2b). The t-threshold technique results in a linear fit (R²>0.98) for all combinations of the tested variables. The curve fitting technique also results in a significant linear fit for all combinations with R² values ranging from 0.91 to 0.98.

TABLE 2
Kinetic assay calibrations using alamarBlue, XTT and turbidity as indicators of cell
viability. The kinetic assays were calibrated in terms of equivalent CFU using both
techniques of data analysis. R² is the correlation coefficient for a linear fit.

| | | | Curve fitting method | | T-threshold method | |
|---|---|---|---|---|---|---|
| | | | [ln(CFU/ml) | | [ln(CFU/ml) | |
| | | | versus ln(X₀)] | | versus t-th] | |
| Mode of | Growth | Inoculum | Dynamic range | | Dynamic range | |
| measurement | Medium | Growth Phase | (CFU/ml) | R² | (CFU/ml) | R² |
| alamarBlue | modified | stationary | 8 × 10⁶ − 60 | 0.975 | 8 × 10⁶ − 60 | 0.993 |
| alamarBlue | modified | exponential | 1.28 × 10⁷ − 50 | 0.930 | 1.28 × 10⁷ − 80 | 0.979 |
| alamarBlue | unmodified | stationary | 8 × 10⁶ − 60 | 0.965 | 8 × 10⁶ − 60 | 0.990 |
| alamarBlue | unmodified | exponential | 1.28 × 10⁷ − 50 | 0.983 | 1.28 × 10⁷ − 50 | 0.991 |
| XTT | modified | stationary | 8 × 10⁶ − 60 | 0.958 | 8 × 10⁶ − 60 | 0.988 |
| XTT | modified | exponential | 1.28 × 10⁷ − 220 | 0.907 | 1.28 × 10⁷ − 50 | 0.991 |
| XTT | unmodified | stationary | 8 × 10⁶ − 60 | 0.974 | 8 × 10⁶ − 60 | 0.986 |
| XTT | unmodified | exponential | 3.1 × 10⁶ − 50 | 0.977 | 1.28 × 10⁷ − 50 | 0.980 |
| Turbidity | modified | stationary | 8 × 10⁶ − 60 | 0.943 | 8 × 10⁶ − 80 | 0.994 |
| Turbidity | modified | exponential | 1.28 × 10⁷ − 80 | 0.923 | 1.28 × 10⁷ − 50 | 0.964 |
| Turbidity | unmodified | stationary | 8 × 10⁶ − 60 | 0.954 | 8 × 10⁶ − 60 | 0.983 |
| Turbidity | unmodified | exponential | 1.28 × 10⁷ − 50 | 0.950 | 1.28 × 10⁷ − 50 | 0.993 |

The curve fitting technique estimates the parameter X₀ in Eq. (1). The magnitude of this constant is dependant on the lag phase of the kinetic data where the signal to noise ratio can be quite low. This could be a likely reason why the curve fitting values yield weaker correlations.

In particular, based on R² values, the alamarBlue and XTT linear fits are relatively better (closer to R²=1) than turbidity. A dynamic range of six orders of magnitude (approximately 50 to 10⁷ CFU) was obtained for all the tested conditions using both techniques of data analysis. The linear calibrations are statistically rigorous over the entire range of experimental variables (Table 2).

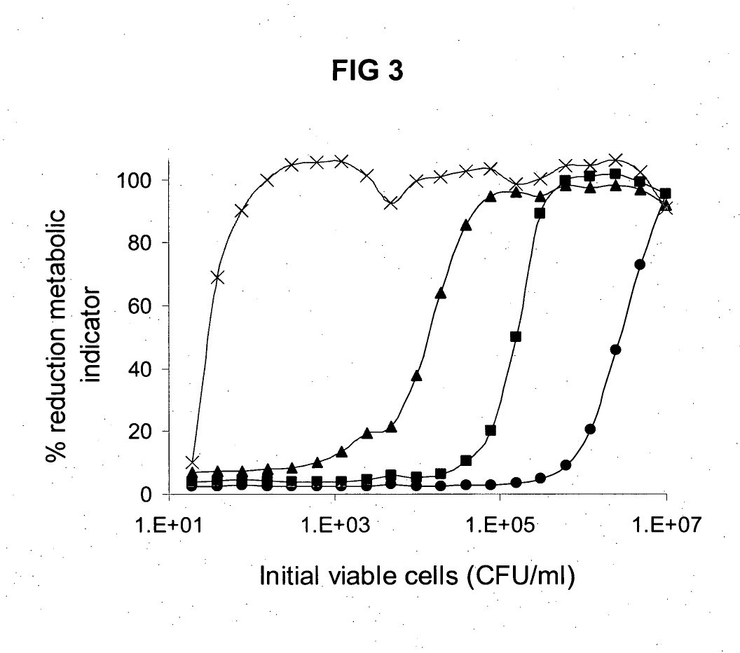

In contrast to the kinetic analysis, when the reduction of the metabolic indictor or turbidity was recorded at endpoints (5 hr, 10 hr, 15 hr or 24 hr) the linear dynamic range was limited to approximately two orders of magnitude (FIG. 3). A detection limit of 1% is the lower estimate of the background absorbance of the metabolic indicator for a standard 96 well plate spectrophotometer (in this case the Biotek Synergy HT™).

### Example 4

### Correlations Between Assays for Cells Exposed to AmB

The results of the KMA were evaluated for planktonic cells by comparing them to the same kinetic analysis based on turbidimetric changes and also with direct CFU measurements. A comparison was made of the ability of each assay to predict viability relative to another. Pearson's correlation coefficients were estimated for each assay combination shown in Tables 3 and 4. Correlations are presented for both techniques of data analysis.

TABLE 3
Correlation between any two assays of interest for the curve fitting technique.

Growth
phase-

| growth | aB vs. XTT | | aB vs. Td | | aB vs. CFU | | XTT vs. Td | | XTT vs. CFU | | Td vs. CFU | |
|---|---|---|---|---|---|---|---|---|---|---|---|---|
| medium | r | p | r | p | r | p | r | p | r | p | r | p |
| Ex-UM | 0.666 | 0.02 | 0.881 | 0.01 | 0.811 | 0.01 | 0.806 | 0.01 | 0.863 | 0.01 | 0.751 | 0.01 |
| (df = 10) | | | | | | | | | | | | |
| St-UM | 0.664 | 0.05 | 0.909 | 0.01 | 0.277 | ns | 0.750 | 0.01 | 0.327 | ns | 0.265 | ns |
| (df = 13) | | | | | | | | | | | | |
| Ex-M | 0.564 | 0.05 | 0.640 | 0.02 | 0.780 | 0.01 | 0.553 | 0.05 | 0.858 | 0.01 | 0.770 | 0.01 |
| (df = 13) | | | | | | | | | | | | |
| St-M | 0.678 | 0.01 | 0.629 | 0.01 | 0.555 | 0.02 | 0.783 | 0.01 | 0.571 | 0.02 | 0.394 | ns |
| (df = 16) | | | | | | | | | | | | |

TABLE 4
Correlation between any two assays of interest for the t-threshold technique.

Growth
phase-

| growth | aB vs. XTT | | aB vs. Td | | aB vs. CFU | | XTT vs. Td | | XTT vs. CFU | | Td vs. CFU | |
|---|---|---|---|---|---|---|---|---|---|---|---|---|
| medium | r | p | r | p | r | p | r | p | r | p | r | p |
| Ex-UM | 0.956 | 0.01 | 0.889 | 0.01 | 0.802 | 0.01 | 0.938 | 0.01 | 0.793 | 0.01 | 0.645 | 0.02 |
| (df = 2) | | | | | | | | | | | | |
| St-UM | 0.805 | 0.01 | 0.810 | 0.01 | −0.12 | ns | 0.884 | 0.01 | −0.45 | ns | −0.51 | ns |
| (df = 12) | | | | | | | | | | | | |
| Ex-M | 0.863 | 0.01 | 0.887 | 0.01 | 0.939 | 0.01 | 0.946 | 0.01 | 0.825 | 0.01 | 0.893 | 0.01 |
| (df = 14) | | | | | | | | | | | | |
| St-M | 0.957 | 0.01 | 0.888 | 0.01 | 0.470 | 0.02 | 0.859 | 0.01 | 0.368 | ns | 0.393 | ns |
| (df = 16) | | | | | | | | | | | | |

r—Pearson's correlation coefficient for comparing parametric data. A two-tailed (df = n − 2) Pearson's
correlation test was used.
df—(degrees of freedom) = n − 2 for a 2-talied test; where n is the number of independent observations.
p—level of significance for a two tailed test.
ns—not significant
Ex—exponential phase,
St—stationary phase
M—modified growth medium,
UM—unmodified growth medium
aB—alamarBlue,
Td—turbidity

The correlations between each combination of the kinetic assays (alamarBlue, XTT and turbidity) were consistently significant for all the conditions tested and for both techniques of data analysis (Table 3 and 4). This implied that there was no significant difference between the XTT and alamarBlue dye when used with C. albicans. Our experimental system used low inoculum (10⁴ CFU/ml) during AmB treatment and the cells were exposed to growth medium during the KMA. Therefore it is. not surprising that the kinetics of metabolic reduction were coupled with cell growth (turbidity) implying that reduction of the metabolic indicator was primarily occurring due to cell proliferation.

In the case of the exponential-phase cells, the correlation between the CFU and kinetic assays (alamarBlue, XTT or turbidity) was significant.. This was the case for both types of growth medium and both techniques of data analysis (Table 3 and 4). On the other hand, for stationary-phase cells and the unmodified medium, the correlation between CFU assay and kinetic assays (alamarBlue, XTT or turbidity) was not significant (Table 3 and 4). These correlations became significant only when a modified medium and the alamarBlue indictor were used. The XTT indicator showed a significant correlation for these conditions only when the curve fitting technique was used for data analysis (Table 4). The kinetic assay based on turbidity did not correlate significantly with the CFU for any of the conditions tested.

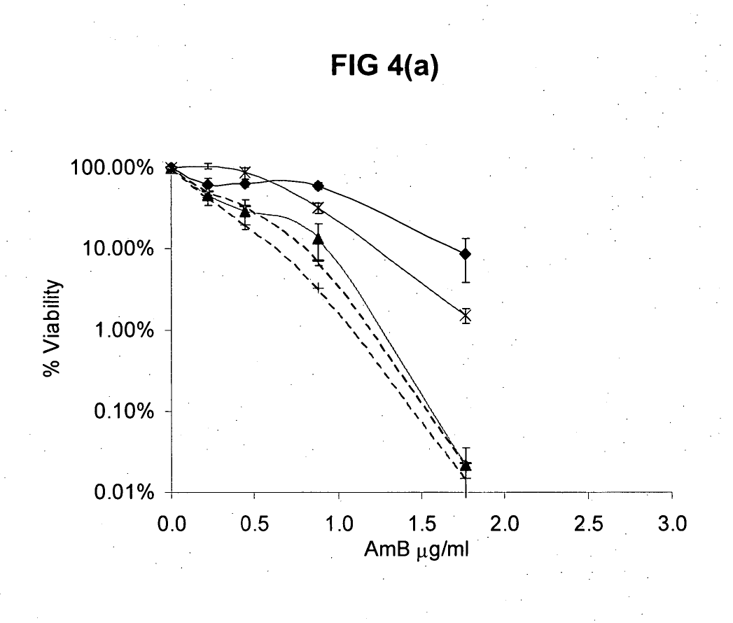

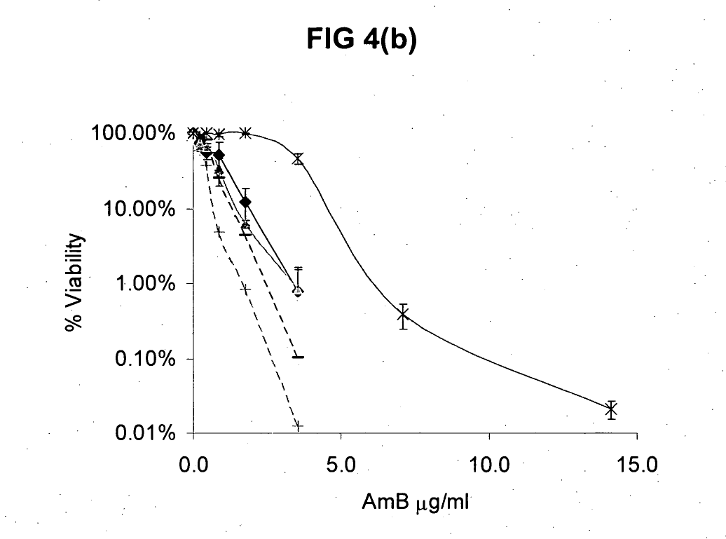

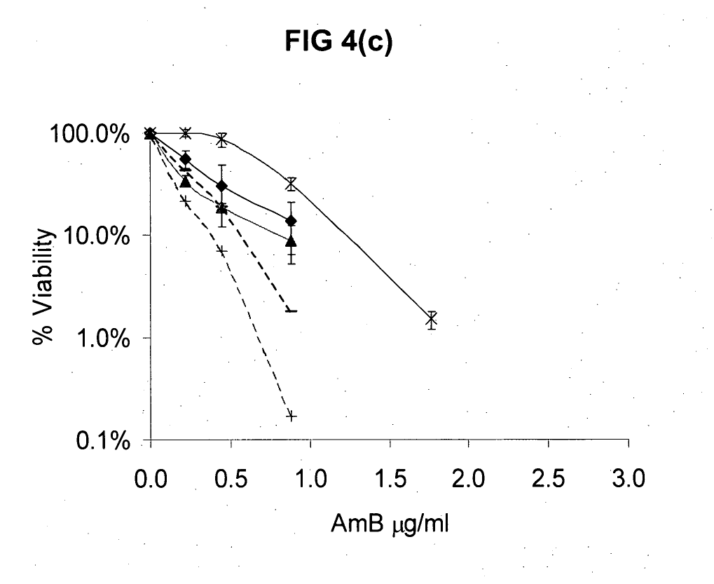

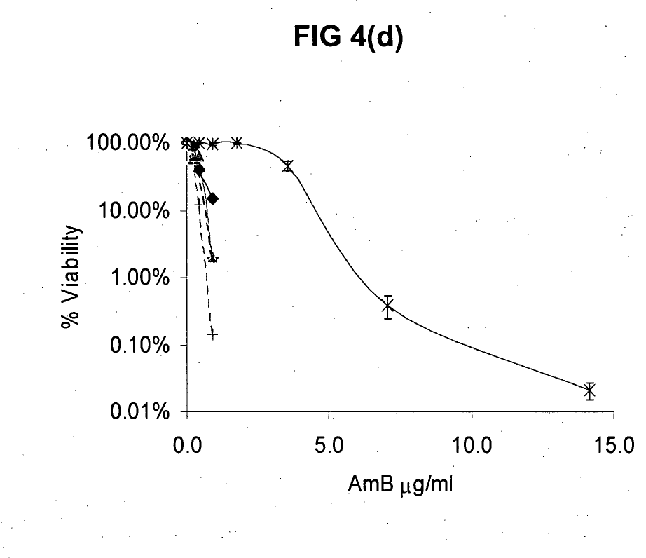

AmB dose response curves for all combinations of variables (except turbidity as the kinetic indicator) are shown in FIG. 4 (a-d). The calibration of the KMA in equivalent CFU offered the added advantage of providing information about how predictions of inhibitory drug concentrations varied between CFU and broth based metabolic assays. This information could be pertinent in analyzing which susceptibility testing method results in better correlation with in vivo antifungal responses. These comparisons can also have implications for the interpretation of results of metabolic assays applied to characterize small resistant subpopulations such as those that might be responsible for Candida albicans biofilm resistance. Based on the planktonic susceptibility data (FIG. 4), it is evident that metabolic assays relatively underestimate AmB resistance of C. albicans when compared with direct CFU measurements.

The two approaches to data analysis were useful in identifying the kinetic parameter that was the strongest representation of viability. Our results show that X₀ from the curve fitting technique consistently predicted a higher estimate of viability at every AmB concentration between the two data analysis techniques (FIG. 4). Based on Eq. (1), the parameter X₀ obtained from the curve fitting technique is proportional to the initial viable cell concentration with the result being that a large X₀ value will result in a shorter lag time in the growth curve (FIG. 2a). A shorter lag time would imply a higher number of initial viable cells (FIG. 1). Our finding that the lag-phase was a more sensitive indicator of viability than the time to reach threshold reduction is consistent with another turbidimetric study of filamentous fungi (29). Although the curve fitting technique had the advantage of being more sensitive in detecting viability, its dependence on data from the lag phase of the kinetic curve made it more prone to be affected by the signal to noise ratio. This effect is apparent in Table 2 where the linear con-elation coefficients (R²) for the curve fitting technique (R² between 0.91 to 098) are lower than those obtained for the t-threshold technique (R²>0.98). In addition, the curve fitting technique also estimates the metabolic reduction or growth rate of every sample (see parameter μ in Eq. (1)) which could be valuable in understanding mechanisms of drug resistance. Such additional information derived from the KMA offers the potential of better correlations with antifungal responses in vivo (because current in vitro antifungal susceptibility tests do not adequately capture antifungal responses in vivo). Therefore the KMA may have good clinical utility.

### Example 5

### Effect of Modified Growth Medium on Correlations Between CFU and Kinetic Assays

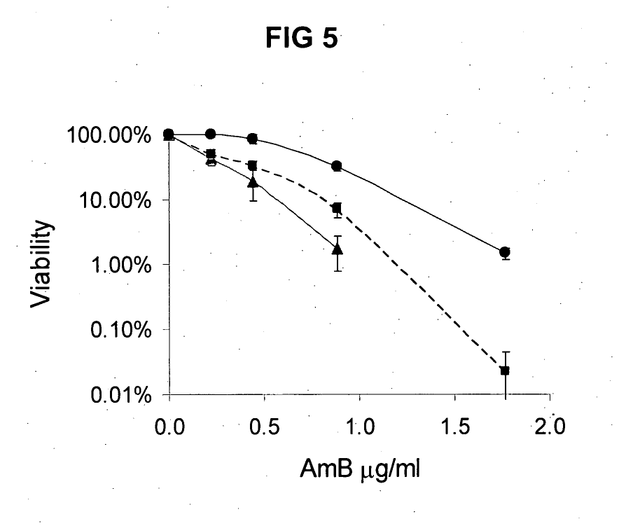

After AmB treatment, the correlations between CFU and kinetic assays weakened (Table 3 and 4). A modified growth medium that quenches AmB action was used in the kinetic assay to study the effect AmB treatment had in perturbing the correlation developed prior to drug treatment. Preliminary experiments with an ergosterol range of 10 μM to 640 μM; MgCl₂ range of 22 μM to 700 μM; and, KCl range of 85 μM to 106 μM, showed that the concentrations of 40 μM ergosterol, 88 μM MgCl₂ and 42.5 μM KCl were the most optimal for maximum detection of viable cells (11, 12, 17). FIG. 5 shows AmB dose response curves for samples that were subjected to both, modified and unmodified medium. Data for the CFU assay applied to the same samples is also shown. The modified medium increased viability estimates at each AmB concentration, this effect is more pronounced at AmB concentration >0.44 μg/ml. It also detects viability up to 1.77 μg/ml AmB unlike the unmodified medium whose detection limit for these conditions was until 0.88 μg/ml. Since the CFU assay consistently yielded a higher estimate of viability, using a modified medium (which recovered more viable cells after AmB treatment) strengthened the correlations between CFU and kinetic assays (Table 3 and 4). Similar effects of the modified medium were observed for the other combination of experimental variables. This indicated it is specifically the AmB treatment that weakened the correlations of CFU with kinetic assays.

There is only a single study that has compared the performance of metabolic reduction with CFU post AmB treatment for C. albicans (45). Tellier et al. (45) used endpoint analysis to compare AmB dose response curves of XTT reduction with CFU, but their assays could detect viable cells only at a cell concentration range of 1×10⁷ to 4×10⁷ CFU/ml. Moreover, their study did not involve C. albicans strains with varying resistance to AmB. Hence, their dose response curves resulted in viability estimates only for AmB concentrations lower than 0.78 μg/ml. Based on qualitative interpretation, they concluded that CFU estimates parallel metabolic reduction. No non linearity between CFU and metabolic reduction in this range of AmB concentration was observed, which is consistent with their results. The KMA, due to its wide dynamic range of detecting viability, enabled our study to explore the aforementioned relationship at higher AmB concentrations. Significant correlation between KMA and CFU for the susceptible exponential phase cells was observed. The two methods significantly correlated up to AmB concentrations of 1.77 μg/ml beyond which complete killing was observed for both methods. For the resistant stationary-phase cells, the KMA did not correlate well with CFU (Table 3 and 4).

It is relevant to note that the accuracy of predicting susceptibility of any one of these assays should be judged only based on comparisons with in vivo antifungal responses. In addition, the limitations of a comparison between CFU and KMA occur because the re-growth of cells occurs in different physical systems i.e., solid agar for CFU versus liquid broth for the metabolic assay. Even though correlations between KMA and CFU were significant for the exponential phase cells, the KMA consistently detected fewer viable cells than the CFU assay. This difference may have occurred because exposure to AmB had damaged some cells in a fashion which temporarily altered their ability to proliferate at normal rates. To test this hypothesis, the media used in the KMA was modified to facilitate faster recovery from the AmB treatment. AmB binds to ergosterol in the cell membrane of C. albicans. This alters the permeability of the membrane causing leakage of ions, eventually leading to cell death (12, 27, 47), Gale (12) proposed a model of AmB action which essentially states that the cell wall lipids constitute a reservoir of AmB that feeds progressively into the membrane structure. This effect remains even when AmB is separated from the cells by centrifugation. This implies that AmB continues to cause damage of cells over a prolonged period. And at higher concentrations of AmB, the progressive damage probably lasts longer. Previous work had suggested that adding optimal concentrations of extracellular ergosterol, K⁺ and Mg²⁺ions considerably reduces or even arrests the damage AmB causes to C. albicans (11, 12, 17). This occurs either by antagonizing the AmB still present in the cell wall by ergosterol, and/or by neutralizing the ionic driving force between the intracellular and extracellular ion concentrations by extracellular K⁺and Mg²⁺. Thus by adding these reagents into the growth medium, the modified medium managed to revive a relatively larger fraction of the surviving cells after AmB treatment. Even though using modified growth medium in the KMA increased the viability estimates, it still could not entirely account for the observed differences with CFU for the resistant stationary-phase cells (FIG. 4). In addition, for the CFU assay, relatively small sized colonies for samples treated with high AmB concentrations was observed when compared with untreated controls. Since the CFU assay is a visual, qualitative assay, a very small or relatively larger colony was still counted as emerging from one surviving cell (irrespective of its size). On the other hand, for the KMA to detect viability, the cells had to re-grow in the liquid broth to the threshold level to generate a reduction signal. Thus each of these methods inherently imposes different physical conditions to generate a viability signal. This could be one simple reason for the observed difference in the case of resistant cells between direct CFU measurements and KMA assays.

### Example 6

### Detection of Acquired Phenotypic Resistance

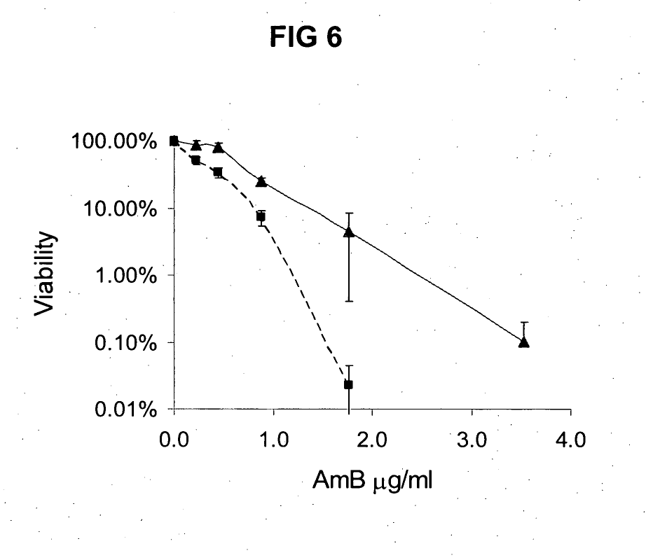

The ability of the kinetic assays (XTT, alamarBlue and turbidity) to detect differences in susceptibilities between exponential-and stationary-phase cells was assessed. As estimated by the broth dilution method, CA-1 exhibited an AmB MIC (0.5 μg/ml) within a standard range for susceptible C. albicans strains. Based on previous studies (12, 18), it was anticipated that stationary-phase cells would exhibit greater AmB resistance than exponential-phase planktonic cells. This difference was evident along the entire AmB dose response curve (FIG. 6). The relative differences in viability become significant as AmB concentrations exceeded 0.44 μg/ml. The stationary phase cells survive up to AmB concentration of 3.55 μg/ml whereas; complete killing of exponential cells is achieved beyond 1.77 μg/ml AmB. The AmB dose response curves shown in FIG. 6 are for the alamarBlue assay; similar differences in AmB resistance were observed when the XTT, turbidity or CFU assays were used. To maintain clarity, curves are only shown for the t-threshold technique. The analysis of data using the curve fitting technique also resulted in the expected differences of AmB resistance.

### Example 7

### KMA for In Situ Biofilm Susceptibility Testing

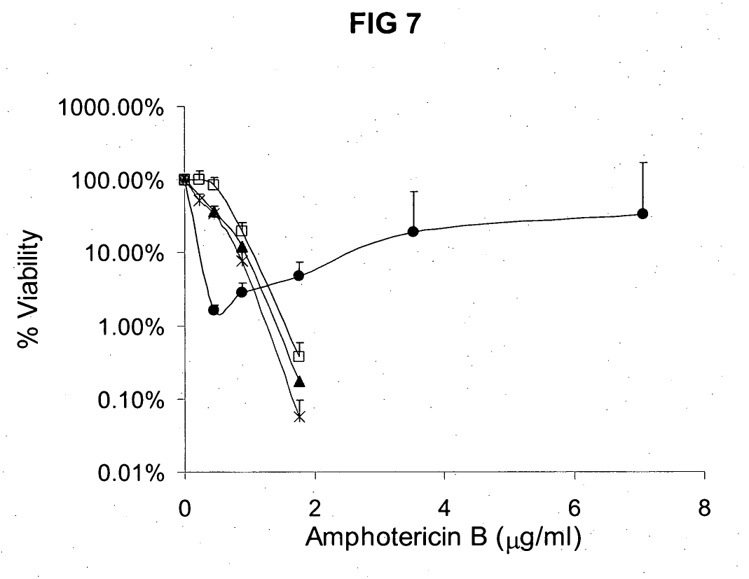

The KMA was preformed in a 96-well plate format for both planktonic and biofilm systems therefore providing a high throughput platformat for in situ susceptibility testing. Since the outer surface of tubing (biofilm was present on the inner side) of the TF snugly fits along the walls of the wells in a 96 well plate, absorbance data could be recorded in real time without perturbing the contents of the well. The KMA (with alamarBlue and modified growth medium) was used for susceptibility testing of C. albicans biofilms to AmB grown in the TF. The t-threshold technique was used to analyze the kinetic data. The large volume of kinetic data could be analyzed within a few minutes using specially written Matlab programs. Exponential-and stationary-phase planktonic cells were tested in parallel to obtain a relative comparison of susceptibility. The dose-response curve for the biofilm subpopulations and planktonic populations used for comparative analysis is shown in FIG. 7. The expected high level of resistance of the basal blastospores subpopulation was observed. At AmB concentrations greater than or equal to 3.7 μg/ml, the basal blastospores were clearly more active than the other three populations. The basal blastospore subpopulation exhibited metabolic activity up to a tested concentration of 28.26 μg/ml AmB (not shown in FIG. 7). Even at this highest AmB concentration, microscopic observation revealed the appearance of budding planktonic yeast forms during the KMA, suggesting that the basal blastospores produced viable cells. At lower concentrations (<1.77 μg/ml), the basal blastospore subpopulation showed a greater loss of metabolic activity than the other samples. This result would be expected if a portion of the cells in the basal blastospore subpopulation were more susceptible to AmB than the other populations and suggests that this subpopulation is itself not completely homogeneous with respect to its susceptibility to AmB.

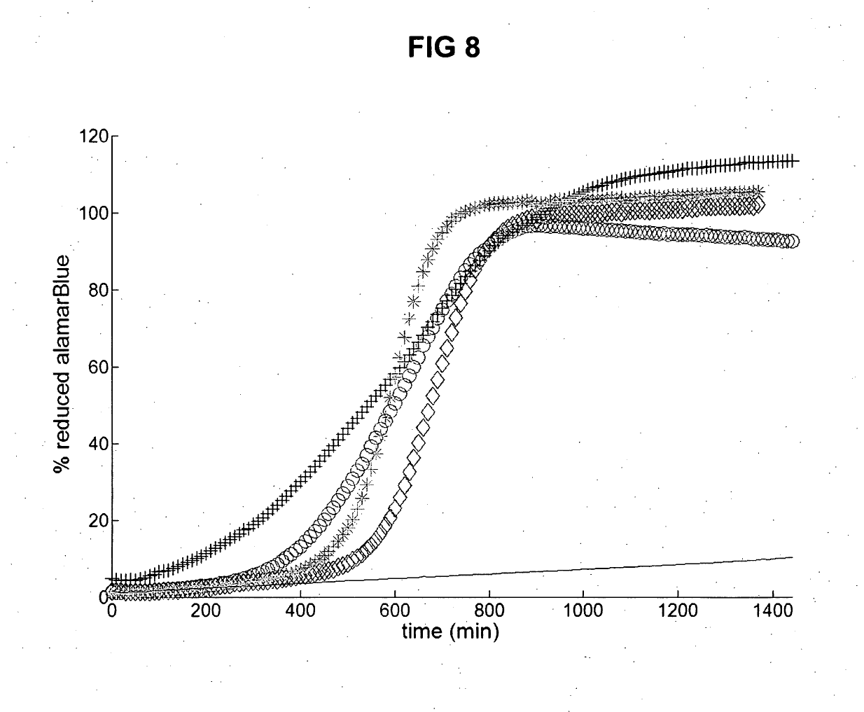

FIG. 8 depicts curves generated from kinetic data averaged for all the samples from three independent experiments. As apparent from the plot, the cures almost overlap. Each kinetic curve represents inoculums used during susceptibility testing. The kinetic parameter (of time for 50 percent reduction) for every cell population was compared with every other cell population by using a student's t-test. There was no significant difference between any of the populations. This result eliminated the effects of using varying degrees of inoculum in potentially skewing results of the susceptibility test.

In conclusion, biofilm resistance may be associated with a small subpopulation of cells (much<10⁵ CFU), which could be detected quantitatively using the KMA. The size of the subpopulation has relevance in understanding biofilm drug resistance mechanisms. Quantitative nature of the KMA could also help control the size of biofilm inoculum used in susceptibility testing. The cell mass to drug ratio can be a major factor affecting the resistance profiles manifested by any drug-organism combination. It is extremely difficult to precisely control the numbers of adhered biofilm cells growing on a specific section of surface. It is argued that biofilm resistance is overestimated because researchers tend to use large numbers of biofilm cells as inoculum. Because the KMA can detect the numbers of equivalent CFU in a biofilm sample, it can help characterize inoculums used in antimicrobial susceptibility testing to eliminate disproportionate ratios of cell mass to drug. The 96 well plate format enabled testing many samples (sections of tubing containing biofilm) simultaneously in each experiment. This feature was helpful to obtain enough number of replicates to make data analysis statistically significant for the inherently heterogeneous nature of biofilm growth. In addition, the KMA inherently normalizes the heterogeneous metabolic states of a biofilm because of its dependence on post drug treatment cell proliferation to reduce the metabolic indicator. This is achieved by forcing the surviving cells to proliferate at the same growth rate independent of their initial metabolic state.

While this invention has been described in certain embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

### REFERENCES

1. Antachopoulos, C., J. Meletiadis, E. Roilides, T. Sein, T. J. Walsh. 2006. Rapid susceptibility testing of medically important zygomycetes by XTT assay. J. Clin. Microbiol. 44: 553-60.
2. Baillie, G. S., L. J. Douglas. 1999. Candida biofilms and their susceptibility to antifungal agents. Methods Enzymol. 310: 644-56.
3. Carmen, J. C., B. L. Roeder, J. L. Nelson, B. L. Beckstead, C. M. Runyan, G. B. Schaalje, R. A. Robison, W. G. Pitt. 2004. Ultrasonically enhanced vancomycin activity against Staphylococcus epidermidis biofilms in vivo. J. Biomater. Appl. 18: 237-45.
4. Chandra, J., D. M. Kuhn, P. K. Mukherjee, L. L. Hoyer, T. McCormick, M. A. Ghannoum. 2001. Biofilm formation by the fungal pathogen Candida albicans: Development, architecture, and drug resistance. J. Bacteriol. 183: 5385-5394.
5. Chandra, J., P. K. Mukherjee, S. D. Leidich, F. F. Faddoul, L. L. Hoyer, L. J. Douglas, M. A. Ghannoum. 2001. Antifungal resistance of Candidal biofilms formed on denture acrylic in vitro. J. Dent. Res. 80: 903-908.
6. Costerton, J. W., P. S. Stewart, E. P. Greenberg. 1999. Bacterial biofilms: A common cause of persistent infections. Science 284: 1318-1322.
7. Cuenca-Estrella, M., J. L. Rodriguez-Tudela. 2001. Present status of the detection of antifungal resistance: the perspective from both sides of the ocean. Clin. Microbiol. Infect. 7 Suppl 2: 46-53.
8. Davies, D. 2003. Understanding biofilm resistance to antibacterial agents. Nature Rev. Drug Discov. 2: 114-122.
9. Ellis, D. 2002. Amphotericin B: spectrum and resistance. J. Antimicrob. Chemother. 49: 7-10.
10. Espinel-Ingroff, A. 2006. Comparison of three commercial assays and a modified disk diffusion assay with two broth microdilution reference assays for testing zygomycetes, Aspergillus spp., Candida spp., and Cryptococcus neoformans with posaconazole and amphotericin B. J. Clin. Microbiol. 44: 3616-22.
11. Gale, E. F. 1974. The release of potassium ions from Candida albicans in the presence of polyene antibiotics. J. Gen. Microbiol. 80: 451-65.
12. Gale, E. F. 1986. Nature and development of phenotypic resistance to amphotericin B in Candida albicans. Adv. Microb. Physiol. 27: 277-320.
13. Groll, A. H., H. Kolve. 2004. Antifungal agents: in vitro susceptibility testing, pharmacodynamics, and prospects for combination therapy. Eur. J. Clin. Microbiol. Infect. Dis. 23: 256-70.
14. Han, Y. M., R. P. Morrison, J. E. Cutler. 1998. A vaccine and monoclonal antibodies that enhance mouse resistance to Candida albicans vaginal infection. Infect. Immun. 66: 5771-5776.
15. Hawser, S. P., L. J. Douglas. 1995. Resistance of Candida albicans biofilms to antifungal agents in-vitro. Antimircrob. Agents and Chemother. 39: 2128-2131.
16. Hawser, S. P., H. Norris, C. J. Jessup, M. A. Ghannoum. 1998. Comparison of a 2,3-bis(2-methoxy-4-nitro-5-sulfophenyl)-5-[(phenylamino)carbonyl]-2H-t etrazolium hydroxide (XTT) colorimetric method with the standardized National Committee for Clinical Laboratory Standards method of testing clinical yeast isolates for susceptibility to antifungal agents. J Clin Microbiol 36: 1450-2.
17. Kerridge, D., T. Y. Koh, A. M. Johnson. 1976. The interaction of amphotericin B methyl ester with protoplasts of Candida albicans. J. Gen. Microbiol. 96: 117-23.
18. Khot, P. D., P. A. Suci, R. L. Miller, R. D. Nelson, B. J. Tyler. 2006. A small subpopulation of blastospores in Candida albicans biofilms exhibit resistance to amphotericin B associated with differential regulation of ergosterol and {beta}-1,6-glucan pathway genes. Antimicrob. Agents and Chemother. 50: 3708-16.
19. Kontoyiannis, D. P., R. E. Lewis. 2002. Antifungal drug resistance of pathogenic fungi. Lancet 359: 1135-44.
20. Kuhn, D. M., M. Balkis, J. Chandra, P. K. Mukherjee, M. A. Ghannoum. 2003. Uses and limitations of the XTT assay in studies of Candida growth and metabolism. J. Clin. Microbiol. 41: 506-8.
21. LaFleur, M. D., C. A. Kumamoto, K. Lewis. 2006. Candida albicans biofilms produce antifungal-tolerant persister cells. Antimicrob Agents Chemother 50: 3839-46.
22. Lamfon, H., S. R. Porter, M. McCullough, J. Pratten. 2004. Susceptibility of Candida albicanis biofilms grown in a constant depth film fermentor to chlorhexidine, fluconazole and miconazole: a longitudinal study. J. Antimicrob. Chemother. 53: 383-385.
23. Latge, J. P. 1999. Aspergillus fumigatus and aspergillosis. Clin. Microbiol. Rev. 12: 310-50.
24. Lewis, K. 2001. Riddle of biofilm resistance. Antimicrob. Agents and Chemother. 45: 999-1007.
25. Lewis, R. E., D. P. Kontoyiannis, R. O. Darouiche, Raad, II, R. A. Prince. 2002. Antifungal activity of amphotericin B, fluconazole, and voriconazole in an in vitro model of Candida catheter-related bloodstream infection. Antimicrob. Agents and Chemother. 46: 3499-3505.
26. Lewis, R. E., N. P. Wiederhold, M. E. Klepser. 2005. In vitro phannacodynamics of amphotericin B, itraconazole, and voriconazole against Aspergillus, Fusarium, and Scedosporium spp. Antimicrob. Agents and Chemother. 49: 945-51.
27. Loeffler, J., D. A. Stevens. 2003. Antifungal drug resistance. Clin. Infect. Dis. 36: S31-41.
28. Meletiadis, J., J. W. Mouton, J. F. Meis, B. A. Bouman, J. P. Donnelly, P. E. Verweij. 2001. Colorimetric assay for antifungal susceptibility testing of Aspergillus species. J. Clin. Microbiol. 39: 3402-8.
29. Meletiadis, J., D. T. te Dorsthorst, P. E. Verweij. 2003. Use of turbidimetric growth curves for early determination of antifungal drug resistance of filamentous fungi. J. Clin. Microbiol. 41: 4718-25.
30. Motulsky, H., 1995. Intuitive Biostatistics. New York: Oxford University Press Inc.
31. Mukherjee, P. K., G. Y. Zhou, R. Munyon, M. A. Ghannoum. 2005. Candida biofilm: a well-designed protected environment. Med. Mycol. 43: 191-208.
32. NCCLS. 1997. Reference method for broth dilution antifungal susceptibility testing of yeasts. NCCLS document M27-A. NCCLS, Wayne, Pa.
33. Neely, M. N., M. A. Ghannoum. 2000. The exciting future of antifungal therapy. Eur J Clin Microbiol Infect Dis 19: 897-914.
34. Perea, S., T. F. Patterson. 2002. Antifungal resistance in pathogenic fungi. Clin. Infect. Dis. 35: 1073-80.
35. Pettit, R. K., C. A. Weber, M. J. Kean, H. Hoffmann, G. R. Pettit, R. Tan, K. S. Franks, M. L. Horton. 2005. Microplate alamar blue assay for, Staphylococcus epidermidis biofilm susceptibility testing. Antimicrob. Agents and Chemother. 49: 2612-7.
36. Pfaller, M. A. 2005. Antifungal susceptibility testing methods. Curr. Drug Targ. 6: 929-43.
37. Pfaller, M. A., A. L. Barry. 1994. Evaluation of a novel colorimetric broth microdilution method for antifungal susceptibility testing of yeast isolates. J. Clin. Microbiol. 32: 1992-1996.
38. Pfaller, M. A., C. Grant, V. Morthland, J. Rhine-Chalberg. 1994. Comparative evaluation of alternative methods for broth dilution susceptibility testing of fluconazole against Candida albicans. J. Clin. Microbiol. 32: 506-9.
39. Ramage, G., K. vande Walle, B. L. Wickes, J. L. Lopez-Ribot. 2001. Standardized method for in vitro antifungal susceptibility testing of Candida albicans biofilms. Antimicrob. Agents and Chemother. 45: 2475-2479.
40. Ramage, G., K. VandeWalle, S. P. Bachmann, B. L. Wickes, J. L. Lopez-Ribot. 2002. In vitro pharmacodynamic properties of three antifungal agents against preformed Candida albicans biofilms determined by time-kill studies. Antimicrob. Agents and Chemother. 46: 3634-3636.
41. Ramage, G., K. Vandewalle, B. L. Wickes, J. L. Lopez-Ribot. 2001. Characteristics of biofilm formation by Candida albicans. Rev. Iberoam. Micol. 18:163-70.
42. Rex, J. H., M. A. Pfaller. 2002. Has antifungal susceptibility testing come of age Clin. Infect. Dis. 35: 982-9.
43. Sanglard, D., F. C. Odds. 2002. Resistance of Candida species to antifungal agents: molecular mechanisms and clinical consequences. Lancet Infect. Dis. 2: 73-85.
44. Stewart, P. S., J. W. Costerton. 2001. Antibiotic resistance of bacteria in biofilms. Lancet 358 :135-138.
45. Tellier, R., M. Krajden, G. A. Grigoriew, I. Campbell. 1992. Innovative endpoint determination system for antifungal susceptibility testing of yeasts. Antimicrob. Agents and Chemother. 36:1619-25.
46. Vianen, W. v., S. de Marie, M. T. ten Kate, R. A. Mathot, I. A. Bakker-Woudenberg. 2006.

Caspofungin: antifungal activity in vitro, pharmacokinetics, and effects on fungal. load and animal survival in neutropenic rats with invasive pulmonary aspergillosis. J. Antimicrob. Chemother. 57: 732-40.
47. White, T. C., K. A. Marr, R. A. Bowden. 1998. Clinical, cellular, and molecular factors that contribute to antifungal drug resistance. Clin. Microbiol. Rev. 11: 382-407.
48. Yamaguchi, H., K. Uchida, K. Nagino, T. Matsunaga. 2002. Usefulness of a colorimetric method for testing antifungal drug susceptibilities of Aspergillus species to voriconazole. J. Infect. Chemother. 8: 374-7.

What is claimed is:

1. A method of determining susceptibility of a biofilm to a molecule of interest, the method comprising:
culturing the biofilm with the molecule of interest;
determining the kinetic profile of a metabolic indicator associated with the biofilm;
determining the number of viable cells in the biofilm;
determining the percentage of surviving cells in the biofilm by comparing the number of viable cells in the biofilm with the number of viable cells in a control culture to which the molecule of interest was not added,
wherein determining the number of viable cells in the biofilm comprises analyzing an exponential phase of the kinetic profile of the metabolic indicator.

2. The method according to claim 1, wherein the biofilm comprises a cell selected from the group consisting of prokaryotic, eukaryotic, bacterial, fungal, plant, animal, mammalian, gram-negative, gram-positive, human, and C. albicans cells.

3. The method according to claim 1, wherein the metabolic indicator is selected from the group consisting of alamarBlue, XTT, florescent dyes, and turbidity.

4. The method according to claim 1, further comprising calibrating the determining the number of viable cells in the biofilm

5. The method according to claim 4, wherein calibrating the determining the number of viable cells in the biofilm comprises comparing the results of the determining the number of viable cells in the biofilm with another viability assay.

6. The method according to claim 5, wherein the other viability assay is selected from the group consisting of colony forming unit assays, hemacytometer counts, total protein level assays, specific protein level assays, and nucleic acid level assays.

7. The method according to claim 4, further comprising determining a correction factor and utilizing the correction factor to correct the results of the determining the number of viable cells in the biofilm.

8. The method according to claim 1, wherein analyzing the state of or change in the metabolic indicator during an exponential phase comprises a method selected from the group consisting of t-threshold and curve fitting.

9. The method according to claim 1, wherein the molecule of interest is an antibiotic, antifungal or cytotoxic compound.

10. The method according to claim 9, wherein the molecule of interest is selected from the group consisting of Penicillin G; D-Cyloserine; Vancomycin; Bacitracin; Cephalosporin C; Tetracycline; Erythromycin; Chloramphenicol; Streptomycin; Nalidixic Acid; Rifampicin; Triethoprim; EDTA; Lysozyme, Amphotericin B; Nystatin; Caspofungin; Mycafungin; Anidulafungin; Fluconazole; Intraconazole; Voriconazole; Posaconazole; Chlorhexidine; Terbinafine; Flucytosine; and Gaiseofulvin.

11. The method according to claim 1, wherein the culturing the biofilm with the molecule of interest and the determining the state of or change in the metabolic indicator over time are performed without disrupting the biofilm.

12. The method according to claim 1 further comprising removing the molecule of interest from the biofilm prior to determining the state of or change in the metabolic indicator over time.

13. The method according to claim 1, further comprising adding to the biofilm molecules or compounds known or thought to inhibit or affect the activity of the molecule of interest before determining the state of or change in the metabolic indicator over time.

14. A method of determining the number of viable cells in a culture, the method comprising:
determining the kinetic profile of a metabolic indicator associated with the culture;
determining the number of viable cells in the culture;
wherein determining the number of viable cells in the culture comprises analyzing the kinetic profile of the metabolic indicator.

15. The method according to claim 14, wherein the kinetic profile of the metabolic indicator comprises a profile selected from the group consisting of exponential, logarithmic, saturation, Monod, linear, polynomial, zero order, first order, and second order kinetics.

16. The method according to claim 14, wherein the culture comprises a cell selected from the group consisting of prokaryotic, eukaryotic, bacterial, fungal, plant, animal, mammalian, gram-negative, gram-positive, human, and C. albicans cells.

17. The method according to claim 14, wherein the metabolic indicator is selected from the group consisting of alamarBlue, XTT, florescent dyes, and turbidity.

18. The method according to claim 14, further comprising calibrating the determining the number of viable cells in the biofilm

19. The method according to claim 14, wherein analyzing the kinetic profile of the metabolic indicator comprises a method selected from the group consisting t-threshold and curve fitting.

20. The method according to claim 14, wherein the culture comprises a cell selected from the group consisting of cell that is floating, planktonic, adherent, part of or associated with a biofilm or other cell cluster or mass, adherent to or otherwise associated with a solid surface, comprises hyphae, and mixtures thereof.
